(19) United States
(12) Patent Application Publication
(10) Pub. No.: US 2011/0201945 A1
(43) Pub. Date: Aug. 18, 2011

(54) HEMODYNAMIC STABILITY DETECTION DURING ARRHYTHMIA USING RESPIRATION SENSOR

(76) Inventors: Dan Li, Shoreview, MN (US); Arjun Sharma, St. Paul, MN (US); Yi Zhang, Plymouth, MN (US)

(21) Appl. No.: 13/027,503

(22) Filed: Feb. 15, 2011

Related U.S. Application Data
(60) Provisional application No. 61/305,258, filed on Feb. 17, 2010.

(51) Int. Cl. A61B 5/0205; A61N 1/365; A61N 1/39

(52) U.S. Cl. 600/484; 607/14; 607/6

(57) ABSTRACT

Detected changes in respiration parameters, either alone or in conjunction with other physiological signals, can be used to discriminate between hemodynamically stable and hemodynamically unstable tachyarrhythmias.

### CROSS-REFERENCE TO RELATED APPLICATIONS

This application claims the benefit of U.S. Provisional Application No. 61/305,258, filed on Feb. 17, 2010, under 35 U.S.C. §119(e), which is incorporated herein by reference in its entirety.

### BACKGROUND

Implantable medical devices (IMDs) are devices designed to be implanted into a patient. Some examples of these devices include cardiac rhythm management (CRM) devices. CRM devices include implantable pacemakers, implantable cardioverter defibrillators (ICDs), and devices that include a combination of pacing and defibrillation including cardiac resynchronization therapy. The devices are typically used to treat patients using electrical therapy and to aid a physician or caregiver in patient diagnosis through internal monitoring of a patient's condition. The devices can include electrical leads in communication with sense amplifiers to monitor electrical heart activity within a patient, and often include sensors to monitor other internal patient parameters. Other examples of implantable medical devices include implantable insulin pumps or devices implanted to administer drugs to a patient.

Additionally, some IMDs detect events by monitoring electrical heart activity signals. By monitoring cardiac signals, IMDs are able to detect abnormally rapid heart rate, or tachyarrhythmia. Although detecting an occurrence of tachyarrhythmia is important, it can be even more helpful if additional physiologic information is known about the tachyarrhythmia, such as if the tachyarrhythmia is hemodynamically stable or unstable. Hemodynamically stable tachyarrhythmia does not cause a significant drop in the patient's blood pressure or cardiac output, and it can generally be treated with anti-tachyarrhythmia pacing (ATP). Hemodynamically unstable tachyarrhythmia, on the other hand, can result in a significant drop in a patient's blood pressure or cardiac output, such that the global or regional perfusion is not adequate to support normal organ function. Hemodynamically unstable tachyarrhythmia generally requires shock therapy or cardioversion. Thus, an IMD that can not only detect tachyarrhythmias, but also discriminate between hemodynamically stable and unstable tachyarrhythmias, can be used to help guide therapy decisions.

### OVERVIEW

This document describes, among other things, systems and methods for discriminating between hemodynamically stable and hemodynamically unstable tachyarrhythmias using detected changes in respiration parameters, either alone or in conjunction with other physiological signals.

Example 1 can include subject matter that can include an apparatus comprising: a cardiac rhythm management device comprising: a tachyarrhythmia detection circuit configured to detect whether tachyarrhythmia is present in a subject; a respiration sensing circuit, coupled to the tachyarrhythmia detection circuit, configured to sense, during the tachyarrhythmia, a respiration signal from the subject; an auxiliary sensing circuit, coupled to the tachyarrhythmia detection circuit, configured to sense, during the tachyarrhythmia, an auxiliary physiological signal from the subject; and a processor circuit, coupled to the respiration sensing circuit and the auxiliary sensing circuit, the processor circuit configured to: determine a characteristic of the respiration signal; determine a characteristic of the auxiliary physiological signal; determine a measure of concordance between the characteristic of the respiration signal and the characteristic of the auxiliary physiological signal; and use the measure of concordance to determine a hemodynamic stability characteristic of the tachyarrhythmia.

In Example 2, the subject matter of Example 1 can optionally include the processor circuit configured to determine the characteristic of the respiration and auxiliary physiological signals, respectively, determined over multiple cardiac cycles.

In Example 3, the subject matter of any one of Examples 1-2 can optionally include the auxiliary sensing circuit configured to sense at least one of a physical activity level, a heart rate, a heart sound, or an acceleration.

In Example 4, the subject matter of any one of Examples 1-3 can optionally include the processor configured to determine the characteristic of the respiration signal by determining at least one of: a time rate of change of a respiration rate, a time rate of change of a respiration depth, or a time rate of change of a respiration morphological pattern; wherein the processor is configured to determine the characteristic of the respiration signal using the characteristic of the respiration signal determined over multiple cardiac cycles.

In Example 5, the subject matter of any one of Examples 1-4 can optionally include the processor configured to: compare the characteristic of the respiration signal to a first threshold value; compare the characteristic of the auxiliary physiological signal to a second threshold value; and use the comparisons to determine an indication of at least one of concordance or discordance.

In Example 6, the subject matter of any one of Examples 1-5 can optionally include the processor configured to determine discordance when the characteristic of the respiration signal exceeds the first threshold value and the characteristic of the auxiliary physiological signal is less than the second threshold value. In Example 7, the subject matter of any one of Examples 1-6 can optionally include the processor configured to declare that the tachyarrhythmia is hemodynamically unstable in response to a determined discordance.

In Example 8, the subject matter of any one of Examples 1-7 can optionally include the processor configured to determine concordance when: 1) the characteristic of the respiration signal exceeds the first threshold value and the characteristic of the auxiliary physiological signal exceeds the second threshold value; or 2) the characteristic of the respiration signal is less than the first threshold value and the characteristic of the auxiliary physiological signal is less than the second threshold value.

In Example 9, the subject matter of any one of Examples 1-8 can optionally include the processor configured to declare that the tachyarrhythmia is hemodynamically stable in response to a determined concordance.

In Example 10, the subject matter of any one of Examples 1-9 can optionally include the hemodynamic stability characteristic of the tachyarrhythmia including one of hemodynamic stability or hemodynamic instability.

In Example 11, the subject matter of any one of Examples 1-10 can optionally include the processor configured to communicate an indication of the hemodynamic stability characteristic of the tachyarrhythmia to a user interface or process.

In Example 12, the subject matter of any one of Examples 1-11 can optionally include the processor configured to use the hemodynamic stability characteristic of the tachyarrhythmia to control therapy provided to the subject.

In Example 13, the subject matter of any one of Examples 1-12 can optionally include a therapy circuit, coupled to the processor circuit, the therapy circuit configured to provide anti-tachyarrhythmia pacing to the subject when the tachyarrhythmia is hemodynamically stable.

In Example 14, the subject matter of any one of Examples 1-13 can optionally include a therapy circuit, coupled to the processor circuit, the therapy circuit configured to provide shock therapy to the subject when the tachyarrhythmia is hemodynamically unstable.

In Example 15, the subject matter of any one of Examples 1-14 can optionally include a therapy circuit, coupled to the processor circuit, the therapy circuit configured to withhold shock therapy when the tachyarrhythmia is hemodynamically stable.

Example 16 can include, or can optionally be combined with any one of Examples 1-15 to include subject matter that can include determining that a tachyarrhythmia is present in a subject; sensing a respiration signal from the subject during the tachyarrhythmia; sensing an auxiliary physiological signal, other than the respiration signal, from the subject during the tachyarrhythmia; determining a characteristic of the respiration signal; determining a characteristic of the auxiliary physiological signal; determining a measure of concordance between the characteristic of the respiration signal and the characteristic of the auxiliary physiological signal; and using the measure of concordance to determine a hemodynamic stability characteristic of the tachyarrhythmia.

In Example 17, the subject matter of any one of Examples 1-16 can optionally include determining the characteristic of the respiration and auxiliary physiological signals, respectively, determined over multiple cardiac cycles.

In Example 18, the subject matter of any one of Examples 1-17 can optionally include sensing at least one of a physical activity level, a heart rate, a heart sound, or an acceleration.

In Example 19, the subject matter of any one of Examples 1-18 can optionally include determining at least one of: a time rate of change of a respiration rate, a time rate of change of a respiration depth, or a time rate of change of a respiration morphological pattern; and wherein determining the characteristic of the respiration signal includes determining the characteristic of the respiration signal determined over multiple cardiac cycles.

In Example 20, the subject matter of any one of Examples 1-19 can optionally include comparing the characteristic of the respiration signal to a first threshold value; comparing the characteristic of the auxiliary physiological signal to a second threshold value; and using the comparisons to determine an indication of at least one of concordance or discordance.

In Example 21, the subject matter of any one of Examples 1-20 can optionally include determining discordance when the characteristic of the respiration signal exceeds the first threshold value and the characteristic of the auxiliary physiological signal is less than the second threshold value.

In Example 22, the subject matter of any one of Examples 1-21 can optionally include declaring that the tachyarrhythmia is hemodynamically unstable in response to a determined discordance.

In Example 23, the subject matter of any one of Examples 1-22 can optionally include determining concordance when: 1) the characteristic of the respiration signal exceeds the first threshold value and the characteristic of the auxiliary physiological signal exceeds the second threshold value; or 2) the characteristic of the respiration signal is less than the first threshold value and the characteristic of the auxiliary physiological signal is less than the second threshold value.

In Example 24, the subject matter of any one of Examples 1-23 can optionally include declaring that the tachyarrhythmia is hemodynamically stable in response to a determined concordance.

In Example 25, the subject matter of any one of Examples 1-24 can optionally include the hemodynamic stability characteristic of the tachyarrhythmia including one of hemodynamic stability or hemodynamic instability.

In Example 26, the subject matter of any one of Examples 1-25 can optionally include communicating an indication of the hemodynamic stability characteristic of the tachyarrhythmia to a user interface or process.

In Example 27, the subject matter of any one of Examples 1-26 can optionally include using the hemodynamic stability characteristic of the tachyarrhythmia to control therapy provided to the subject.

In Example 28, the subject matter of any one of Examples 1-27 can optionally include providing anti-tachyarrhythmia pacing to the subject when the tachyarrhythmia is hemodynamically stable.

In Example 29, the subject matter of any one of Examples 1-28 can optionally include providing shock therapy to the subject when the tachyarrhythmia is hemodynamically unstable.

In Example 30, the subject matter of any one of Examples 1-29 can optionally include withholding shock therapy when the tachyarrhythmia is hemodynamically stable.

Example 31 can include, or can optionally be combined with any one of Examples 1-30 to include subject matter that can include an apparatus comprising: a cardiac rhythm management device comprising: a tachyarrhythmia detection circuit configured to detect whether tachyarrhythmia is present in a subject; a respiration sensing circuit, coupled to the tachyarrhythmia detection circuit, configured to sense a respiration signal from the subject; and a processor circuit, coupled to the respiration sensing circuit, the processor circuit configured to: determine a characteristic of a respiration rate or interval from the respiration signal; compare the characteristic of the respiration rate or interval to a specified threshold value; when the characteristic of the respiration rate exceeds the specified threshold value, declare that the tachyarrhythmia is hemodynamically unstable.

These examples can be combined in any permutation or combination. This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a schematic diagram illustrating generally an example of an implantable or other ambulatory cardiac rhythm management (CRM) device.

FIG. 2 is a block diagram illustrating generally an example of portions of the CRM device electronics unit.

FIGS. 3A and 3B illustrate examples of conceptualized (not real) data that can be used to determine a relationship, such as a measure of concordance, between respiratory rate and an auxiliary physiological signal.

FIG. 4 illustrates an example of a method for differentiating between stable and unstable tachyarrhythmia.

FIG. 5 illustrates an example of another method for differentiating between stable and unstable tachyarrhythmia.

### DETAILED DESCRIPTION

The present inventors have recognized, among other things, that changes in respiration parameters, in conjunction with other physiological signals, can be used to discriminate between hemodynamically stable and hemodynamically unstable tachyarrhythmias. Respiration signals can be detected by minute ventilation sensors or other respiration sensors. Discrimination of hemodynamically stable versus unstable tachyarrhythmias can help guide therapy decisions.

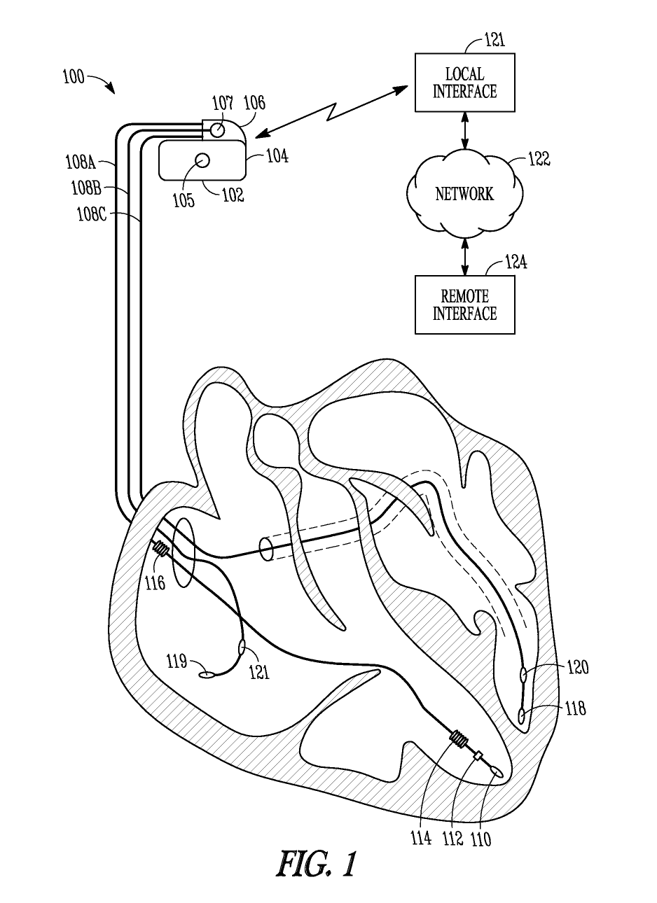

FIG. 1 shows an example of an implantable or other ambulatory cardiac rhythm management (CRM) device 100. In an example, the CRM device 100 can include an electronics unit 102 that can include a hermetically-sealed biocompatible housing 104 and a header 106 extending therefrom. The housing 104 can carry a power source and electronics. The header 106 can include one or more receptacles, such as for receiving the proximal ends of intravascular leads 108A-C. In an example, the lead 108A can be an intravascular RV lead that can extend from the superior vena cava (SVC) into the right atrium (RA), and then into the right ventricle (RV). The lead 108A can include an RV apical tip electrode 110, a slightly more proximal RV ring electrode 112, a still slightly more proximal RV shock coil electrode 114, and an even more proximal RA or SVC shock coil electrode 116. The various electrodes can be used for delivering electrical energy or sensing intrinsic electrical heart signals. An intravascular CS/LV lead 108C can extend from the SVC into the RA, through a coronary sinus (CS) into the coronary vasculature, such as near a portion of a left ventricle (LV). In an example, this second CS/LV lead 108B can include at least a distal electrode 118 and a proximal electrode 120, from which electrostimulation energies can be delivered or intrinsic electrical heart signals can be sensed. An intravascular right atrial (RA) lead 108B can extend from the SVC into the RA, and can include a distal electrode 119 and a proximal electrode 121. Other electrodes (e.g., a housing electrode 105 on the housing 104, a header electrode 107 on the header 106, an epicardial electrode, a subcutaneous electrode located away from the heart, or an electrode located elsewhere) or leads can be used.

In an example, an implantable CRM device 100 can include a communication circuit, such as to wireless communicate unidirectionally or bidirectionally with an external local interface 121, such as a CRM device programmer, repeater, handheld device, or the like. The local interface 121 can be configured to communicate via a wired or wireless computer or communication network 122 to a remote interface 124, such as a remote computer or server or the like.

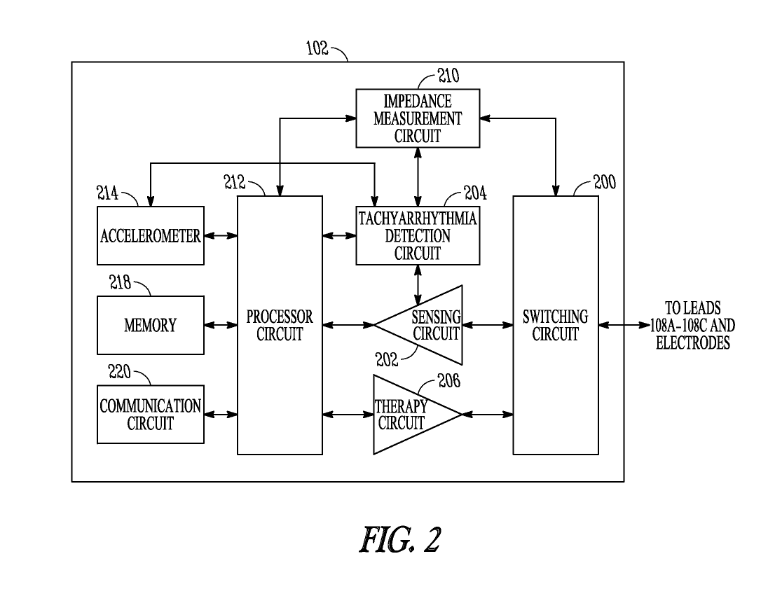

FIG. 2 shows an example of portions of the CRM device electronics unit 102. In an example, this can include a switching circuit 200, such as for selectively connecting to the various electrodes such as on the leads 108A-C or elsewhere. A sensing circuit 202 can be selectively coupled to various electrodes by the switching circuit 200, and can include sense amplifiers, filter circuits, other circuits such as for sensing intrinsic electrical signals, such as intrinsic heart signals. The sensing circuit 202 can be coupled to a tachyarrhythmia detection circuit 204. The tachyarrhythmia detection circuit 204 can be configured to detect tachyarrhythmia in a patient, such as by using heart rate or morphology information from the depolarizations sensed by the sensing circuit 202. A therapy circuit 206 can be selectively coupled to various electrodes by the switching circuit 200, and can include pacing energy generation circuitry (e.g., capacitive, inductive, or other) such as for generating, storing, or delivering an electrostimulation, cardioversion, defibrillation, or other energy. An impedance measurement circuit 210 can be selectively coupled to various electrodes by the switching circuit 200, such as for measuring a lead impedance, a tissue impedance, a regional or organ impedance, or other impedance. Impedance measurements, such as transthoracic impedance measurements, can be used, for example, to detect a respiration signal, such as respiration rate, respiration depth, or a respiration morphological pattern. Impedance measurements can also be used to detect a fluid status. The impedance measurement circuit 210 can be coupled to the tachyarrhythmia detection circuit 204. In an example, the sensing circuit 202, the tachyarrhythmia detection circuit 204, the therapy circuit 206, or the impedance measurement circuit 210 can be coupled to a processor circuit 212. In an example, the processor 212 can perform instructions, such as for signal processing of signals derived by the sensing circuit 202, the tachyarrhythmia detection circuit 204, or the impedance circuit 210, or for controlling operation of the therapy circuit 206 or other operations of the CRM device 100. The processor 212 can be coupled to or include a physical activity sensor, such as an accelerometer 214, configured to sense a patient's acceleration or physical activity level. The accelerometer 214 can be coupled to the tachyarrhythmia detection circuit 204. In an example, the accelerometer 214 can be configured to sense other measures of physical activity, such as heart sounds. In an example, the accelerometer 214 can be configured to detect respiration signals by detecting muscle stretch or movement of the diaphragm or intercostal muscles. Although not shown in FIG. 2, other sensors, in addition to the accelerometer 214 and the impedance measurement circuit 210, can be configured to detect respiration signals. Examples of such other sensors configured to detect respiration signals include acoustic or ultrasound sensors, as well as blood pressure or blood flow sensors. The processor 212 can also be coupled to or include a memory circuit 218, such as for storing or retrieving instructions or data, or a communication circuit 220, such as for communicating with the local interface 121.

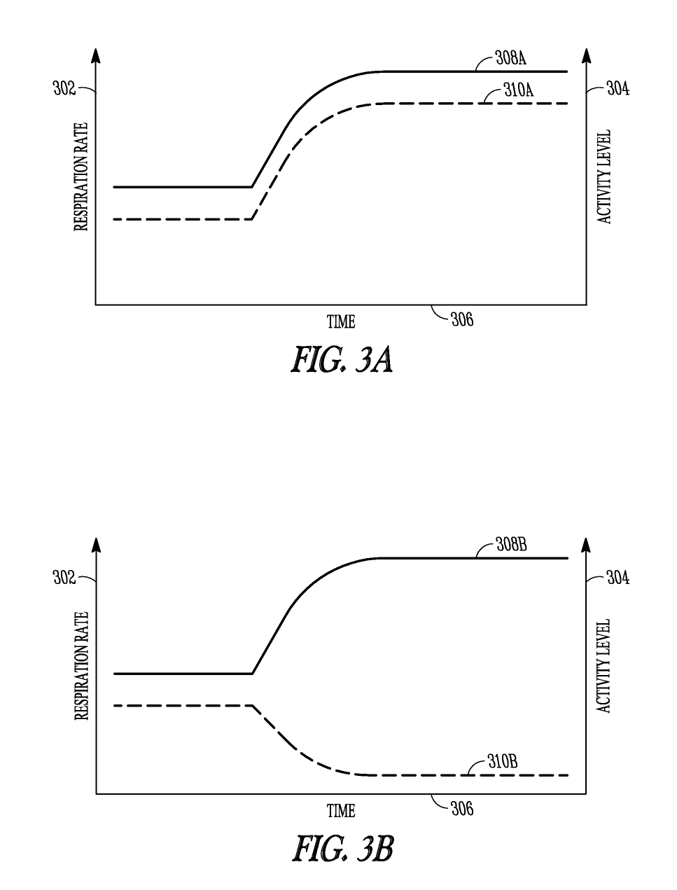

FIGS. 3A and 3B illustrate examples of conceptualized (not real) data that can be used to determine a relationship, such as a measure of concordance, between respiratory signal, such as respiratory rate, and an auxiliary physiological signal, such as physical activity level. Other respiratory signals that can be used, instead of or in addition to respiratory rate, include respiration depth or respiration morphological pattern. Other auxiliary physiological signals that can be used, instead of or in addition to physical activity level, include heart rate, heart sounds, or acceleration, for example. In the examples illustrated, the relationships between respiratory rate and physical activity level can be used to differentiate hemodynamically stable tachyarrhythmia from hemodynamically unstable tachyarrhythmia. As described with respect to FIG. 2 above, a respiration signal can be detected, for example, by the impedance measurement circuit 210 or the accelerometer 214, and the physical activity level can be detected by the accelerometer 214. In an example, by comparing the time rate of change of the respiratory rate to the time rate of change of the physical activity level, a measure of concordance can be determined. In FIGS. 3A and 3B, y-axis 302 represents respiratory rate and y-axis 304 represents physical activity level. Both respiratory rate and physical activity level are plotted against time, which is represented by the x-axis 306. In FIG. 3A, respiratory rate, represented by line 308A, increases concordantly with activity level, represented by line 310A. Concordance of respiratory rate and activity level can be observed under normal physiological conditions, such as during exercise. Concordance of respiratory rate and activity level during a tachyarrhythmia can be indicative of a hemodynamically stable tachyarrhythmia. In FIG. 3B, respiratory rate, represented by line 308B, increases while activity level, represented by line 310B, decreases. The resulting discordance of respiratory rate and activity level can be indicative of a hemodynamically unstable tachyarrhythmia, which, in turn, can result in hemodynamic deterioration.

In an example, the relationship, indicative of a hemodynamic stability characteristic, between a characteristic of a respiration signal and a characteristic of a physical activity level, can be defined as an interdependence between the characteristic of the respiration signal and the characteristic of the physical activity level. Thus, for example, in addition to or instead of a measure of concordance, a relationship can be determined between respiratory rate and physical activity level by comparing a respiration rate corresponding to a specified activity level to a threshold value. In an example, a respiration rate that is above a threshold value for a specified activity level can be indicative of a hemodynamically unstable tachyarrhythmia. Likewise, a respiration rate that is below a threshold value for a specified activity level can be indicative of a hemodynamically stable tachyarrhythmia.

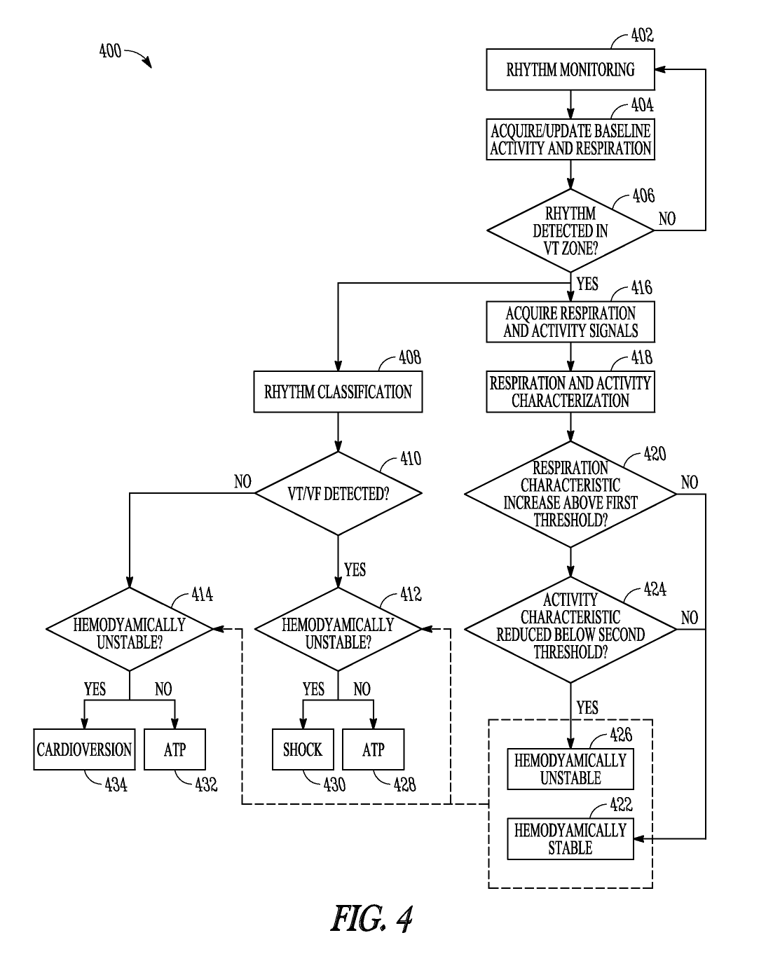

FIG. 4 illustrates an example of a method 400 for differentiating between stable and unstable tachyarrhythmia. At 402, cardiac rhythm is detected and monitored, such as by an implantable CRM device. At 404, baseline respiration and physical activity level measurements are acquired or updated. In an example, other auxiliary physiological signals, such as heart rate, heart sounds, or posture, can be monitored in addition to or instead of physical activity. At 406, if the detected cardiac rhythm is in the ventricular tachyarrhythmia zone, such as 120 to 200 beats per minute (bpm), then at 408, the detected tachyarrhythmia is classified. The tachyarrhythmia can be classified, for example, via analysis of the detected electrophysiological signal. At 410, if the tachyarrhythmia is classified as either ventricular tachyarrhythmia or ventricular fibrillation, then the process flows to 412. In an example, when the tachyarrhythmia is classified as ventricular fibrillation (e.g., >200 bpm), the hemodynamic stability determination at 412 can be skipped, and the process can flow directly to 430, where shock therapy is delivered to the patient. At 410, if the tachyarrhythmia is not classified as either ventricular tachyarrhythmia or ventricular fibrillation, then the process flows to 414.

At 406, if the detected cardiac rhythm is in the ventricular tachyarrhythmia zone, then at 416 respiration and physical activity signals are acquired during the tachyarrhythmia. Respiration and activity signals can be acquired at 416 concurrently with classification of the tachyarrhythmia at 408. In an example, respiration and activity signals can be acquired at 416 continuously. In an example, respiration and activity signals can be acquired at 416 only if specified respiration sensor activation conditions are met. In an example, a condition for respiration sensor activation can be a detected heart rate that is below the lower limit of the ventricular tachyarrhythmia zone but above a specified threshold value. This can allow for the detection and treatment of slow ventricular tachyarrhythmias. Another example of a condition for respiration sensor activation can include a cardiac cycle length stability that is below a specified threshold value, such as 20 milliseconds. Still other examples of conditions for respiration sensor activation include the presence of specified electrocardiogram characteristics, the status of therapy delivery (e.g. anti-tachyarrhythmia pacing), a detected drop in intracardiac impedance that is below a specified threshold value, or manual activation by a user or healthcare provider.

At 418, the respiration and activity signals are characterized. In an example, characterization of the respiration and activity signals can occur on an ongoing basis. In an example, characterization of the respiration and activity signals can occur only if specified respiration sensor activation conditions are met, such as those described above. The respiration signal can be characterized, for example, by determining a time rate of change of a respiration rate, a time rate of change of a respiration depth, or a time rate of change of a respiration morphological pattern. In an example, the respiration signal can be characterized by determining a change in correlation or a change in coherence among multiple respiration signal measurements within a specified number of heart beats. The physical activity signal can be characterized, for example, by determining a physical activity level, a heart rate, a heart sound, or an acceleration. In an example, the respiration and physical activity signals can be characterized over multiple cardiac cycles. At 420, if the respiration characteristic is not increased above a first threshold value, then at 422 the tachyarrhythmia is declared hemodynamically stable. At 420, if the respiration characteristic is increased above the first threshold value, then the process flows to 424. In an example, the respiration characteristic can be measured and compared to the first threshold value after a specified time period, such as 5-10 seconds from the onset of the tachyarrhythmia. At 424, if the activity characteristic is not reduced below a second threshold, then at 422 the tachyarrhythmia is declared hemodynamically stable. At 424, if the activity characteristic is reduced below the second threshold, then at 426, the tachyarrhythmia is declared hemodynamically unstable. In an example, the activity characteristic can be measured and compared to the second threshold value after a specified time period, such as 5-10 seconds from the onset of the tachyarrhythmia. After the tachyarrhythmia has been declared hemodynamically stable at 422 or unstable at 426, the process flows back to 412 and 414. At 412, if the tachyarrhythmia has been declared hemodynamically stable, then at 428 anti-tachyarrhythmia pacing (ATP) is provided to the patient. At 412, if the tachyarrhythmia has been declared hemodynamically unstable, then at 430 shock therapy is provided to the patient. At 414, if the tachyarrhythmia has been declared hemodynamically stable, then at 432 ATP is provided to the patient. At 414, if the tachyarrhythmia has been declared hemodynamically unstable, then at 434 cardioversion is provided to the patient.

In an example, at 416, a respiration signal can be acquired without acquiring an activity signal. At 418, the respiration signal can be characterized, such as by determining a respiration rate or interval, a tidal volume, a depth of respiration, or a respiratory pattern. In an example, the respiration signal can be characterized by determining a time rate of change or a relative change of a respiration rate, a tidal volume, a respiration depth, or a respiratory pattern. At 420, the respiration characteristic can be compared to a specified threshold value. If the respiration characteristic exceeds the specified threshold value, then at 426, the tachyarrhythmia is declared hemodynamically unstable. If the respiration characteristic does not exceed the threshold value, then at 422 the tachyarrhythmia is declared hemodynamically stable. In an example, at 420, the respiration characteristic can be compared to a specified criteria instead of, or in addition to, being compared to a specified threshold value. If the respiration characteristic meets the specified criteria, then at 426, the tachyarrhythmia is declared hemodynamically unstable. If the respiration characteristic does not meet the specified criteria, then at 422 the tachyarrhythmia is declared hemodynamically stable. After the tachyarrhythmia has been declared hemodynamically stable at 422 or unstable at 426, the process flows back to 412 and 414, as described above.

An example of a situation in which the respiration characteristic alone (e.g., without a physical activity characteristic) can be used to distinguish hemodynamically stable and unstable tachyarrhythmias includes when the patient's detected heart rate is above a specified threshold value (e.g. 180 bpm). In this situation, it is unlikely that physical activity would result in or contribute to such a high heart rate. Therefore, it would be unnecessary to detect the physical activity characteristic in conjunction with the respiration characteristic in order to determine hemodynamic stability or instability; rather, analysis of the respiration characteristic alone would suffice to differentiate hemodynamically stable and unstable tachyarrhythmias. For example, a respiration rate that is increased above a specified threshold can be indicative of hemodynamically unstable tachyarrhythmia, and a respiration rate that is not increased above a specified threshold can be indicative of hemodynamically stable tachyarrhythmia.

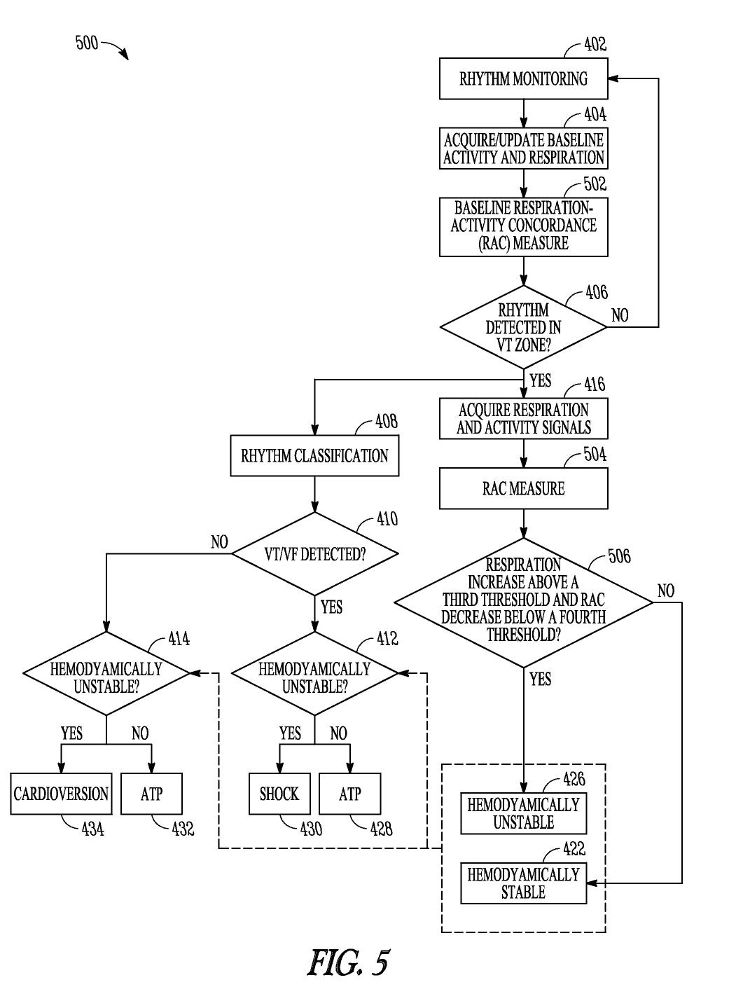

FIG. 5 illustrates an example of another method 500 for differentiating between stable and unstable tachyarrhythmia. At 402, cardiac rhythm is detected and monitored, such as by an implantable CRM device. At 404, baseline respiration and physical activity level measurements are acquired or updated. In an example, other auxiliary physiological signals, such as heart rate, heart sounds, or posture, can be monitored in addition to or instead of physical activity. At 502, a baseline Respiration-Activity Concordance (RAC) measure is obtained. The RAC measure can be obtained, for example, using plotted data such as that shown in FIGS. 3A and 3B. In an example, the RAC measure can be a ratio of the respiratory rate measured at a specified time to the activity level measured at the same specified time. In an example, the RAC measure can be a ratio can be a ratio of the rate of change of the respiratory rate to the rate of change of the activity level. At 406, if the detected cardiac rhythm is in the ventricular tachyarrhythmia zone, such as 120 to 200 bpm, then at 408, the detected tachyarrhythmia is classified. The tachyarrhythmia can be classified, for example, via analysis of the detected electrophysiological signal. At 410, if the tachyarrhythmia is classified as either ventricular tachyarrhythmia or ventricular fibrillation, then the process flows to 412. In an example, when the tachyarrhythmia is classified as ventricular fibrillation (e.g., >200 bpm), the hemodynamic stability determination at 412 can be skipped, and the process can flow directly to 430, where shock therapy is delivered to the patient. At 410, if the tachyarrhythmia is not classified as either ventricular tachyarrhythmia or ventricular fibrillation, then the process flows to 414.

At 406, if the detected cardiac rhythm is in the ventricular tachyarrhythmia zone, then at 416 respiration and physical activity signals are acquired during the tachyarrhythmia. Respiration and activity signals can be acquired at 416 concurrently with classification of the tachyarrhythmia at 408. In an example, respiration and activity signals can be acquired at 416 continuously. In an example, respiration and activity signals can be acquired at 416 only if specified respiration sensor activation conditions are met. In an example, a condition for respiration sensor activation can be a detected heart rate that is below the lower limit of the ventricular tachyarrhythmia zone but above a specified threshold value. This can allow for the detection and treatment of slow ventricular tachyarrhythmias. Another example of a condition for respiration sensor activation can include a cardiac cycle length stability that is below a specified threshold value, such as 20 milliseconds. Still other examples of conditions for respiration sensor activation include the presence of specified electrocardiogram characteristics, the status of therapy delivery (e.g. anti-tachyarrhythmia pacing), a detected drop in intracardiac impedance that is below a specified threshold value, or manual activation by a user or healthcare provider.

At 504, the RAC measure is determined during the tachyarrhythmia. In an example, determination of the RAC measure can occur on an ongoing basis. In an example, determination of the RAC measure can occur only if specified respiration sensor activation conditions are met, such as those described above with respect to FIG. 4. In an example, the RAC measure can be determined after a specified time period, such as 5-10 seconds after the onset of the tachyarrhythmia. In an example, the RAC measure can be determined over multiple cardiac cycles. At 506, if the respiration characteristic is not increased above a third threshold value, or if the RAC measurement is not decreased such that it is below a fourth threshold value, then at 422 the tachyarrhythmia is declared hemodynamically stable. At 506, if the respiration characteristic is increased above a third threshold value, and if the RAC measurement is decreased such that it is below a fourth threshold value, then at 426 the tachyarrhythmia is declared hemodynamically unstable. In an example, the fourth threshold value can be a specified percentage of the baseline RAC measure, such as 80%. After the tachyarrhythmia has been declared hemodynamically stable at 422 or unstable at 426, the process flows back to 412 and 414. At 412, if the tachyarrhythmia has been declared hemodynamically stable, then at 428 anti-tachyarrhythmia pacing (ATP) is provided to the patient. At 412, if the tachyarrhythmia has been declared hemodynamically unstable, then at 430 shock therapy is provided to the patient. At 414, if the tachyarrhythmia has been declared hemodynamically stable, then at 432 ATP is provided to the patient. At 414, if the tachyarrhythmia has been declared hemodynamically unstable, then at 434 cardioversion is provided to the patient.

### Additional Notes

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

The claimed invention is:

1. An apparatus comprising:
a cardiac rhythm management device comprising:
a tachyarrhythmia detection circuit configured to detect whether tachyarrhythmia is present in a subject;
a respiration sensing circuit, coupled to the tachyarrhythmia detection circuit, configured to sense a respiration signal from the subject;
an auxiliary sensing circuit, coupled to the tachyarrhythmia detection circuit, configured to sense an auxiliary physiological signal from the subject; and
a processor circuit, coupled to the respiration sensing circuit and the auxiliary sensing circuit, the processor circuit configured to:
determine a characteristic of the respiration signal;
determine a characteristic of the auxiliary physiological signal;
determine relationship between the characteristic of the respiration signal and the characteristic of the auxiliary physiological signal; and
use the relationship to determine a hemodynamic stability characteristic of the tachyarrhythmia.

2. The apparatus of claim 1, wherein the auxiliary sensing circuit is configured to sense at least one of a physical activity level, a heart rate, a heart sound, or an acceleration.

3. The apparatus of claim 1, wherein the processor is configured to determine the characteristic of the respiration signal by determining at least one of: a time rate of change of a respiration rate, a time rate of change of a respiration depth, or a time rate of change of a respiration morphological pattern;
and wherein the processor is configured to determine the characteristic of the respiration signal using the characteristic of the respiration signal determined over multiple cardiac cycles.

4. The apparatus of claim 1, wherein the processor is configured to:
compare the characteristic of the respiration signal to a first threshold value;
compare the characteristic of the auxiliary physiological signal to a second threshold value; and
use the comparisons to determine an indication of at least one of concordance or discordance.

5. The apparatus of claim 4, wherein the processor is configured to determine discordance when the characteristic of the respiration signal exceeds the first threshold value and the characteristic of the auxiliary physiological signal is less than the second threshold value; and
wherein the processor is configured to declare that the tachyarrhythmia is hemodynamically unstable in response to a determined discordance.

6. The apparatus of claim 4, wherein the processor is configured to determine concordance when: 1) the characteristic of the respiration signal exceeds the first threshold value and the characteristic of the auxiliary physiological signal exceeds the second threshold value; or 2) the characteristic of the respiration signal is less than the first threshold value and the characteristic of the auxiliary physiological signal is less than the second threshold value; and
wherein the processor is configured to declare that the tachyarrhythmia is hemodynamically stable in response to a determined concordance.

7. The apparatus of claim 1, wherein the processor is configured to communicate an indication of the hemodynamic stability characteristic of the tachyarrhythmia to a user interface or process.

8. The apparatus of claim 1, wherein the processor is configured to use the hemodynamic stability characteristic of the tachyarrhythmia to control therapy provided to the subject.

9. The apparatus of claim 8, comprising a therapy circuit, coupled to the processor circuit, the therapy circuit configured to provide anti-tachyarrhythmia pacing to the subject when the tachyarrhythmia is classified as ventricular tachyarrhythmia and is hemodynamically stable.

10. The apparatus of claim 8, comprising a therapy circuit, coupled to the processor circuit, the therapy circuit configured to:
provide shock therapy to the subject when the tachyarrhythmia is classified as ventricular tachyarrhythmia and is hemodynamically unstable; and
withhold shock therapy when the tachyarrhythmia is classified as ventricular tachyarrhythmia and is hemodynamically stable.

11. A method comprising:
determining that a tachyarrhythmia is present in a subject;
sensing a respiration signal from the subject;
sensing an auxiliary physiological signal, other than the respiration signal, from the subject;
determining a characteristic of the respiration signal;
determining a characteristic of the auxiliary physiological signal;
determining a relationship between the characteristic of the respiration signal and the characteristic of the auxiliary physiological signal; and
using the relationship to determine a hemodynamic stability characteristic of the tachyarrhythmia.

12. The method of claim 11, wherein sensing an auxiliary physiological signal includes sensing at least one of a physical activity level, a heart rate, a heart sound, or an acceleration.

13. The method of claim 11, wherein determining the characteristic of the respiration signal includes determining at least one of: a time rate of change of a respiration rate, a time rate of change of a respiration depth, or a time rate of change of a respiration morphological pattern; and
wherein determining the characteristic of the respiration signal includes determining the characteristic of the respiration signal determined over multiple cardiac cycles.

14. The method of claim 11, wherein determining the relationship includes:
comparing the characteristic of the respiration signal to a first threshold value;
comparing the characteristic of the auxiliary physiological signal to a second threshold value; and
using the comparisons to determine an indication of at least one of concordance or discordance.

15. The method of claim 14, comprising:
determining discordance when the characteristic of the respiration signal exceeds the first threshold value and the characteristic of the auxiliary physiological signal is less than the second threshold value; and
declaring that the tachyarrhythmia is hemodynamically unstable in response to a determined discordance.

16. The method of claim 14, comprising:
determining concordance when: 1) the characteristic of the respiration signal exceeds the first threshold value and the characteristic of the auxiliary physiological signal exceeds the second threshold value; or 2) the characteristic of the respiration signal is less than the first threshold value and the characteristic of the auxiliary physiological signal is less than the second threshold value; and
declaring that the tachyarrhythmia is hemodynamically stable in response to a determined concordance.

17. The method of claim 11, comprising communicating an indication of the hemodynamic stability characteristic of the tachyarrhythmia to a user interface or process.

18. The method of claim 11, comprising using the hemodynamic stability characteristic of the tachyarrhythmia to control therapy provided to the subject.

19. The method of claim 18, comprising providing anti-tachyarrhythmia pacing to the subject when the tachyarrhythmia is hemodynamically stable.

20. The method of claim 18, comprising:
providing shock therapy to the subject when the tachyarrhythmia is hemodynamically unstable; and
withholding shock therapy when the tachyarrhythmia is hemodynamically stable.
